(19) United States
(12) Patent Application Publication
(10) Pub. No.: US 2008/0095826 A1
(43) Pub. Date: Apr. 24, 2008

(54) Method of Increasing Peripheral Blood Lymphocytes

(76) Inventors: Hisayuki Uneyama, Kawasaki-shi (JP); Miki Tomoe, Munakata-shi (JP); Misako Kawai, Kawasaki-shi (JP); Naoki Hayashi, Shizuoka-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/860,600

(22) Filed: Sep. 25, 2007

(30) Foreign Application Priority Data
Oct. 24, 2006 (JP) 289259/2006

(51) Int. Cl. A61K 31/195; A61K 39/00; A61K 47/00

(52) U.S. Cl. 424/439; 424/184.1; 514/561

(57) ABSTRACT

The present invention provides a method of increasing the lymphocyte count in peripheral blood and a method for stimulating the immune system in a subject, which include administering an effective amount of glutamic acid or a salt thereof to the subject. Since glutamic acid or a salt thereof is not toxic and is free of side effects, it is safe, can be presented in the form of food, and is suitable for application to the elderly and subjects with reduced resistance. Since immunity is enhanced, general conditions can be improved and the mind and body can be activated.

This application claims priority under 35 U.S.C. §119 to Japanese Patent Application No. 2006-289259, filed Oct. 24, 2006, the entirety of which is incorporated by reference.

### BACKGROUND OF THE INVENTION

1. Field of the Invention

The present invention relates to a method for stimulating the immune system in a subject by administering an effective amount of glutamic acid or a salt thereof. More particularly, the present invention relates to a method of increasing the lymphocyte count in the peripheral blood in a subject.

2. Brief Description of the Related Art

Lymphocytes are cells which act on the immune system. When there is a significant decrease of the lymphocytes in the peripheral blood, an infection with bacteria, viruses, fungi, parasites, and the like, may result. Repeat infections, frequent abnormal responses to usually benign infectious factors, and infections with rare microorganisms can also occur. For example, an infection with Pneumocystis carinii, cytomegaloviruses, measles, parasites, and the like can easily occur. Pneumonia may develop as a result of such infections, and can sometimes be fatal; therefore these infections pose a serious problem for the elderly, feeble individuals, poorly nourished individuals, those with cancer, and the like.

Conditions which are accompanied by a decrease in the lymphocyte count in the peripheral blood include a nutritional deficiency, radiation therapy, extreme stress, immunosuppressive therapy, inherited lymphocytopenia (e.g., agammaglobulinemia, DiGeorge malformation, Wiskott-Aldrich syndrome, severe composite immunodeficiency syndrome, ataxia telangiectasia, and the like), viral infections (e.g., HIV, granulomatous infection, Hodgkin's lymphoma, and the like), acute bacterial and/or fungal infections, protein-losing gastroenteropathy, and the like.

In developing countries in regions such as South East Asia, Africa, Central and South America, and the like, many people are still living in a chronically nutritionally deficient state. In these regions, one of the problems to be preferentially solved from the aspect of public hygiene is to provide tools to these people so they can defend against infections. In addition, the HIV (Human Immunodeficiency Virus) infection rate is high in these developing regions, and the development of a treatment for delaying the decrease in the peripheral lymphocyte count in conjunction with an antiviral agent is in great demand.

In contrast, the more advanced countries and regions enjoy the benefit of prolonged average life expectancy due to advanced medical progress. However, an important problem that arises in the care and treatment of the elderly who live in assisted-living facilities, hospitals, and the like, is protection from bacterial, fungal, or viral infections which result from a chronic decrease in the circulating lymphocyte count. Maintaining an aseptic environment is the chief method for dealing with opportunistic infections which result from decreased circulating lymphocyte count in cancer patients under radiation therapy or anticancer drug treatments, patients with immune system diseases and transplant patients undergoing immunosuppressive therapy, and patients undergoing palliative care with administration of gamma globulin. Therefore, there is a need in the art for the development of significant preventive methods that improve the circulating lymphocyte count.

There are various immunoenhancing agents which are known to treat subjects with compromised immune function, including those which function by enhancing lymphocyte activation. These known agents include polysaccharides from mushroom extracts (e.g., krestin, lentinan and the like), plant extracts of Viscum album (e.g., mistletoe), and polygamma-glutamic acid (JP-A-2005-187427). These immunoenhancing substances increase the activity of macrophages, T cells, and NK cells, as well as enhance immunity by promoting the production of various cytokines, such as interferon and interleukin-2. Other popular immune-enhancing substances which are generally available include Ginseng Radix (e.g., Ginseng Radix Rubre, Sun Ginseng and the like), garlic, bamboo salt, beans, fermentation products, green sap, various vegetables, organic germanium, various yeasts, elm bark, various marine plants, and the like. All of these general immunoenhancing substances potentiate immunity by enhancing lymphocyte activation, rather than by increasing the lymphocyte count in the peripheral blood.

Alternatively, increasing the blood cell component of the peripheral blood, transplanting bone marrow, and administering various hematopoietic factors are methods which have also been used to potentiate immune function. For example, human granulocyte colony-stimulating factor (G-CSF) is used to increase granulocytes, such as neutrophils and the like, erythropoietin (EPO) is used to increase red blood cells, and thrombopoietin (TPO) is used to increase platelets. Various cytokines (e.g., interleukin, interferon, and the like) have been mostly used in attempts to increase the lymphocyte count in peripheral blood. However, these cytokines not only demonstrate a hematopoietic action but also simultaneously demonstrate various physiologic actions as side effects (e.g., chills, thrill, fever, dysosteogenesis, and the like). Therefore, the use of cytokines is limited, and also they are expensive to produce and formulate as a biopharmaceutical product.

In addition, nutrients have also been used in an attempt to increase the peripheral lymphocyte count, specifically by improving protein nutrient content and using glutamine (amino acid) (Newsholme P, J Nutr. 2001 September; 131 (9 Suppl): 2515S-22S). However, glutamine is expensive to produce, and has some undesirable properties for the perspective of formulating into an acceptable preparation, for example, it is unstable in aqueous solutions. Thus, the development of a functional agent able to be formulated into a stable preparation which can be distributed to many geographic areas at a lower cost is in demand.

The lymphocyte proliferation effect of glutamine is attributable to the direct action of glutamine as an energy source during lymphocyte proliferation, the starting material of nucleic acids, and the like (Newsholme P, J Nutr. 2001 September; 131 (9 Suppl): 2515S-22S). Furthermore, glutamine needs to reach the bone marrow, lymph nodes, and the like, to be effective to enhance in vivo lymphocyte proliferation.

However, since the addition of glutamic acid, which corresponds to glutamine hydrolysate, to a lymphocyte culture supernatant is not known to enhance cell proliferation similar to glutamine, and taking glutamic acid with food is known to not increase the level of glutamic acid or glutamine concentration in the blood (Glutamic acid: Advances in biochemistry and physiology, Edited by L. J. Filer et al., Raven Press (New York) 1979), the abililty of glutamic acid to increase the peripheral blood lymphocyte count could not have been predicted based on the physiological activity and mechanism of action glutamine.

### SUMMARY OF THE INVENTION

The present invention provides a novel, economical, and stable method of increasing the lymphocyte count in peripheral blood.

The present inventors have found that glutamic acid or a salt thereof has a peripheral blood lymphocyte-increasing action, is effective to stimulate the immune system in a subject, and acts as an immunostimulator.

The present invention provides the following:

It is an aspect of the present invention to provide a method of stimulating the immune system in a subject comprising administering an effective amount of glutamic acid or a salt thereof to said subject.

It is a further aspect of the present invention to provide the method as described above, further comprising observing an increase in the lymphocytes present in the peripheral blood of said subject.

It is a further aspect of the present invention to provide the method as described above, wherein the amount of glutamic acid or a salt thereof administered to said subject per day is 75-10,000 mg, based on glutamic acid.

It is a further aspect of the present invention to provide a method of increasing the lymphocytes in the peripheral blood of a subject, comprising administering an effective amount of glutamic acid or a salt thereof to said subject.

It is a further aspect of the present invention to provide the method as described above, wherein the amount of glutamic acid or a salt thereof administered to said subject per day is 75-10,000 mg, based on glutamic acid.

It is a further aspect of the present invention to provide an immunostimulator comprising glutamic acid or a salt thereof and acceptable excipients, diluents, and/or fillers.

It is a further aspect of the present invention to provide the immunostimulator as described above, which functions by increasing the lymphocytes in the peripheral blood of a subject.

It is a further aspect of the present invention to provide the immunostimulator as described above, comprising 75-10,000 mg, based on glutamic acid.

It is a further aspect of the present invention to provide a pharmaceutical composition comprising the immunostimulator as described above.

It is a further aspect of the present invention to provide a food composition comprising the immunostimulator as described above.

It is a further aspect of the present invention to provide an agent for increasing the lymphocytes in peripheral blood comprising glutamic acid or a salt thereof.

It is a further aspect of the present invention to provide the food composition as described above, wherein the food has specified health uses.

It is a further aspect of the present invention to provide a package comprising the food composition as described above, and written matter stating that the food can be taken for the purpose of stimulating immunity or increasing the lymphocytes in the peripheral blood, and explaining the method of administering.

According to the methods of the present invention, a hematopoietic effect is provided by the administration of glutamic acid or a salt thereof, and the resulting decrease of the lymphocyte count in the peripheral blood can be reversed, and therefore, increased. As a result, immunity can be stimulated, and not only the ability to defend against infection is enhanced, but also the energy of mind and body can be recovered. As a result, the general quality of life (QOL) of the patients can be improved. Since glutamic acid or a salt thereof is not toxic, and is free of side effects, it can be administered for a long period of time to a sick person and/or the elderly for the purpose of prophylaxis or treatment.

A salt of glutamic acid is stable for a long time in an aqueous solution or against a certain heat, and can be used in any form upply. Hence, the salt is extremely advantageous as compared to the glutamine described in the aforementioned Newsholme P, J Nutr. 2001 September; 131 (9 Suppl): 2515S-22S.

### BRIEF DESCRIPTION OF THE DRAWINGS

FIG. 1 shows changes in the peripheral lymphocyte count when rice gruel containing monosodium glutamate was taken by a subject.

FIG. 2 shows the profile of the dose of the PEG agent containing monosodium glutamate, and changes in the peripheral lymphocyte count and C-reactive protein.

FIG. 3 shows the results of Hasegawa Dementia Scale-Revised before and 2 months after the intake by a subject of rice gruel containing monosodium glutamate.

### DETAILED DESCRIPTION OF THE EXEMPLARY EMBODIMENTS

In the present invention, glutamic acid may be either in the form of a free acid or a salt thereof. The salt may be either inorganic or organic. Examples of inorganic salts include salts with alkali metals, such as sodium, potassium, and the like, and salts with alkaline earth metals, such as calcium, magnesium, and the like. Examples of organic salts include salts with organic amines, such as ammonia, monoethanolamine, diethanolamine, triethanolamine, and the like, salts with basic amino acids, such as arginine, lysin, and the like, salts with inorganic acids such as hydrochloric acid, hydrobromic acid, nitric acid, sulfuric acid, phosphoric acid, and the like, salts with organic acids such as formic acid, oxalic acid, acetic acid, lactic acid, tartaric acid, citric acid, fumaric acid, malonic acid, methanesulfonic acid, and the like. A particular kind of these salts may be used, or two or more kinds thereof may be used. Due to their ready availability and ease of handling, and the like, salts with alkali metals such as sodium, potassium, and the like, salts with organic amines, and salts with basic amino acids are preferable.

Glutamic acid or the salt thereof may be a hydrate.

In the method of increasing the lymphocytes in the peripheral blood and the method of stimulating the immune system, glutamic acid or a salt thereof may be administered, for example, in the form of an immunostimulator to be described in detail below.

In the present invention, the term “immunostimulator” encompasses pharmaceutical agents and/or compositions and food compositions. Food compositions include nutritionally beneficial foods and foods for specified health uses, and the like. The immunostimulator includes a package containing the pharmaceutical agent or food of the present invention, and written matter stating that the pharmaceutical agent or food is to be administered or taken to stimulate the immune system or to increase the lymphocyte count in peripheral blood, and also stating the method of administration or intake.

The immunostimulator of the present invention can be mixed with an appropriate excipient, an appropriate diluent, and the like, such as those generally used in pharmacological compositions. The immunostimulator, or the mixture thereof, can be prepared by a conventional method to be in the form of a solid or liquid oral dosage form, such as a powder, granule, tablet, capsule, suspension, emulsion, syrup, aerosol, and the like, and/or an external preparation, a suppository, or a sterile injection.

As the carrier, excipient, and/or diluent, lactose, dextrose, sucrose, sorbitol, mannitol, xylitol, maltitol, starch, glycerol, gum acacia, alginate, gelatin, calcium sulfate, calcium silicate, cellulose, methylcellulose, microcrystalline cellulose, polyvinylpyrrolidone, water, methylhydroxybenzoate, propylhydroxybenzoate, talc, magnesium stearate and mineral oil can be used.

In addition, the diluent and excipient may include a filler, extender, binder, wetting agent, disintegrant, surfactant, and the like, or any other agents which are generally used in such a preparation.

The solid dosage form for oral administration includes a tablet, pill, powder, granule, capsule, and the like. The solid dosage form can be prepared by adding at least one excipient, for example, starch, calcium carbonate, sucrose or lactose, gelatin, and the like, to the aforementioned glutamic acid or the salt thereof. Besides simple excipients, a lubricant such as magnesium stearate and talc can also be used. As a liquid preparation for oral administration, a suspension, internal liquid, emulsion, syrup, and the like can be used, which may contain various excipients, such as a wetting agent, sweetening agent, aromatic, preservative, and the like. Water and liquid paraffin are frequently used as simple diluents. The preparation for parenteral administration includes a sterile aqueous solution, non-water-soluble preparation, suspension, emulsion, freeze-dry preparation, and suppository. For the non-water-soluble preparation and suspension, plant oil such as propylene glycol, polyethylene glycol and olive oil, injectable ester such as ethyloleate, and the like, can be used. As the base of a suppository, hard fat (witepsol), macrogol, Tween 61, cacao butter, laurisilva fat, glycerogelatin, and the like can be used.

Glutamic acid or a salt thereof can be prepared as a food, such as foods for the sick, nutritionally-beneficial food, food for specified health uses, and the like. When prepared as a food, the amount of glutamic acid or a salt thereof in the food is 0.008-30 wt %, preferably 0.4-10 wt %, relative to the total weight of the food based on glutamic acid. When the food is a beverage, the amount of glutamic acid is about 0.008-10 g (0.008-10 wt %), preferably about 0.08-2 g, relative to 100 mL of the beverage. The food may be a powder, granule, tablet, capsule, cookie, jelly, beverage, or general food.

The food composition of the present invention may contain general food materials as a base and, for example, various nutritional supplements, such as vitamins, mineral substances (electrolyte), minerals, synthetic flavors, natural flavors, colorants, fillers (cheese, chocolate, and the like), pectic acids or a salt thereof, alginic acids or a salt thereof, organic acids, protective colloid thickeners, pH adjusting agents, stabilizers, preservatives, glycerol, alcohols, and the like.

When the food is in the form of a beverage, it may contain fruit pulp and grain which results from the production of natural fruit juices. The beverage may also be a fruit juice beverage, grain and/or vegetable beverage, or a carbonated beverage containing carbonation agents. The components in the beverage other than glutamic acid and a salt thereof are not particularly limited and, various flavors, natural carbohydrates, and the like can be added as additional components. Natural carbohydrates may include, for example, general saccharides such as monosaccharides (glucose, fructose and the like), disaccharides (maltose, sucrose and the like) and polysaccharides (dextrin, cyclodextrin and the like), as well as sugar alcohols such as xylitol, sorbitol, erythritol and the like. Flavors other than those mentioned above may include natural flavors (thaumatin, extract of stevia (rebaudioside A), glycyrrhizin and the like) and synthetic flavors (saccharin, aspartame and the like).

These materials and components can be used independently or in combination. While the proportion of such additives is not limited, it is generally in the range of about 0.01-20 wt % per 100 wt % of the beverage composition. The proportion of the aforementioned natural carbohydrate is generally about 1-20 g (1-20 wt %), preferably about 5-12 g, per 100 mL of the composition.

When the food is in the form of a powder, granule, tablet, or capsule, it can be processed into a preparation using the additive, and using known methods for formulating pharmaceutical products.

The pharmaceutical composition and food composition of the present invention can be a part of a package also containing written matter stating that the pharmaceutical composition or food composition can be administered or taken for the purpose of stimulating the immune system or increasing lymphocytes in the peripheral blood, and also stating the method of administration or intake, and the like.

The glutamic acid or a salt thereof is administered in an effective amount for a human and/or other animals in need of enhanced immunity (e.g., cattle, horse, sheep, goat, pig, dog, cat, domestic fowl, and the like). When administering to animals, an effective amount of glutamic acid or a salt thereof may be added to the feed. To enhance immunity against infectious diseases, glutamic acid or a salt thereof may be added to the feed within the range of 0.008-95 wt %, preferably 0.08-80 wt %, relative to the total weight of the feed composition based on glutamic acid.

The pharmaceutical composition or food composition of the present invention may also contain, besides glutamic acid or a salt thereof, glutamine and the like, other peripheral blood lymphocyte-increasing agents, or a known immunoenhancing substance, which is known to potentiate immunity by enhancing lymphocyte activation, though it may not increase the lymphocyte count in peripheral blood. Furthermore, any of these additives may be used alone or in combination. Immunoenhancing substances known to potentiate immunity by enhancing lymphocyte activation include polysaccharides from a mushroom extract (krestin, lentinan and the like), plant extracts of Viscum album (mistletoe), polygamma-glutamic acid, Ginseng Radix (Ginseng Radix Rubre, Sun Ginseng and the like), garlic, bamboo salt, beans, fermentation products, green sap, various vegetables, organic germanium, various yeasts, elm bark, various marine plants, and the like.

Combining these conventional immunoenhancing substances enables greater enhancement of immunity because of the synergistic effect provided thereby. For example, cytokines are known to have problematic side effects, therefore, combining the administration of cytokines with glutamic acid or a salt thereof can increase the lymphocyte count in the peripheral blood, reduce the amount of cytokine needed, and reduce the side effects.

While the dose or amount of glutamic acid or a salt thereof will vary depending on the age, sex, body weight, pathology of the subject, and chosen administration pathway, it is generally 8-300 mg/kg, preferably 40-200 mg/kg, based on glutamic acid, for an adult subject per day, which can be administered at one time, or in several portions. The dose or amount is preferably about 0.4 g-3 g.

When the dosage amount is less than 8 mg/kg, the total lymphocyte count in peripheral blood will not increase sufficiently, and when it is more than 300 mg/kg, the effect cannot be enhanced sufficiently to outweigh the difficulty in dosing and increase in cost.

In the present invention, the amount of glutamic acid or a salt thereof for an adult per day is preferably 75-10,000 mg, more preferably 2,000-10,000 mg, based on glutamic acid.

The aforementioned doses are not limited, but can be determined appropriately by those skilled in the art. Since glutamic acid or a salt thereof per se is not toxic or exhibits side effects, it can be administered over a long period of time for the purpose of preventing a decrease in the lymphocyte count in the peripheral blood, preventing infectious diseases, and the like.

The subject to be administered with glutamic acid or a salt thereof is generally healthy, but may be at risk to develop infectious diseases due to a low total lymphocyte count in the peripheral blood, or the subject is elderly or sick, or has already developed an infectious disease and is in need of treatment. The etiology of decreased lymphocyte count in the peripheral blood, followed by the onset of an infectious disease, and the like includes, but is not limited to, nutritional deficiency, radiation therapy, extreme stress, immunosuppressive therapy, inherited lymphocytopenia (agammaglobulinemia, DiGeorge malformation, Wiskott-Aldrich syndrome, severe composite immunodeficiency syndrome, ataxia telangiectasia and the like), viral infection (HIV, granulomatous infection, Hodgkin's lymphoma and the like), acute bacterial or fungal infection, protein-losing gastroenteropathy, and the like.

The normal level of total lymphocytes in the peripheral blood is 1000-4800/μL for an adult, and the total lymphocytes in blood include both T-lymphocytes and B-lymphocytes. About 65% of T-lymphocytes are CD4+ (helper) T-lymphocytes, and it is known that most patients with lymphocytopenia have a decreased absolute count of T-lymphocytes, particularly a decrease in their CD4+ T-lymphocyte count. In the present invention, therefore, healthy subjects and patients with a total lymphocyte count in the peripheral blood below this level are candidates for glutamic acid administration. It is also possible to prophylactically administer glutamic acid or a salt thereof as described herein to healthy subjects and/or patients having a total lymphocyte count within the normal range, but with an upcoming event which may decrease their total lymphocyte count, such as patients scheduled for a medical procedure that may reduce the lymphocyte count, and/or healthy subjects and patients possibly exposed to a a high level of stress.

The present invention is explained in more detail in the following by referring to Examples and Production Example, which are not to be construed as limiting.

### EXAMPLES

### Example 1

Monosodium glutamate monohydrate was added to rice gruel to 0.5 wt %.

Hospitalized elderly test subjects (n=11 (2 males, 9 females), average age of 85.8±8.2, average body weight of 39.7±5.7 kg), were fed the above-mentioned rice gruel with glutamic acid 3 times a day for 2 months, establishing a continuous administration of 0.8 g on average of monosodium glutamate monohydrate per meal.

From 1 month before the start of administration to 2 months after the start of administration, the eating ratio (ratio of weight of food actually eaten to weight of provided food) was measured. Blood samples were collected early in the morning immediately before the start of administration, 1 month after the start of adminstration, 2 months after the start of administration, and 1 month after the completion of administration, and the blood indices were measured. In addition, the body weight was measured.

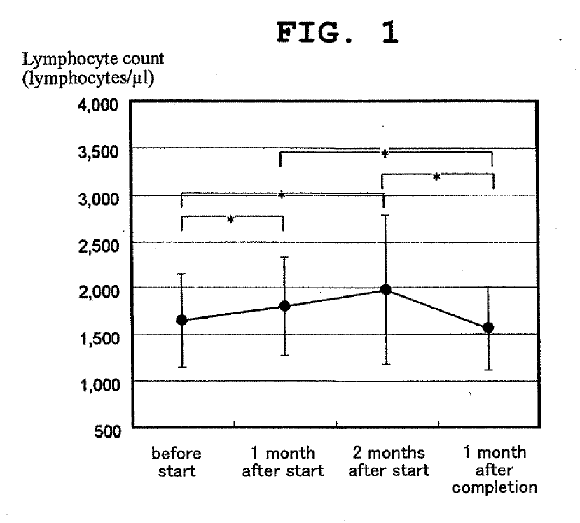

There was no significant difference in the eating ratio after the start of administration as compared to that before the start of administration for both the principal food of rice gruel containing monosodium glutamate monohydrate and the side dish. The energy intake amount and the nutrient intake amount also showed no changes. The blood index and albumin value were also free of significant change, but the peripheral lymphocyte count increased significantly (Friedman test, p<0.05) (FIG. 1). At this time, the C-reactive protein value did not change.

Furthermore, at 1 month after the completion of administration, the peripheral lymphocyte count significantly decreased as compared to during administration (Friedman test, p<0.05) (FIG. 1).

From these results, it can be seen that, even when the energy intake amount and the nutrient intake amount are of the same level, once monosodium glutamate is taken, the peripheral lymphocyte count significantly increases.

### Example 2

Table 1 shows the composition of 4,000 g of enteral nutrient (PEG agent) containing glutamic acid.

TABLE 1

| | | amount | |
|---|---|---|---|
| name of material | | used | unit |
| milk protein source material (casein 66.8 wt %) | | 416 | g |
| monosodium glutamate (1 hydrate) | | 24 | g |
| dextrin | | 1040 | g |
| granulated sugar | | 68 | g |
| water-soluble food fiber | | 117 | g |
| sodium phosphate | | 10.4 | g |
| potassium phosphate | | 9.2 | g |
| magnesium chloride | | 23.6 | g |
| calcium lactate | | 12.8 | g |
| potassium citrate | | 24 | g |
| sodium gluconate | | 80 | g |
| sodium ferrous citrate | | 0.84 | g |
| *mineral yeast Mix | | 3.3 | g |
| **vitamin Mix | | 2.6 | g |
| sodium ascorbate | | 2.2 | g |
| sodium erythorbate | | 2.2 | g |
| flavor | | 4 | g |
| edible fat and oil | | 213 | g |
| emulsifier | | 11.2 | g |
| *mineral | zinc yeast | 1492 | mg |
| yeast Mix | copper yeast | 748 | mg |
| | selenium yeast | 266.8 | mg |
| | manganese yeast | 724 | mg |
| | iodine yeast | 92.4 | mg |
| **vitamin | vitamin A powder (175000 IU/g) | 167.6 | mg |
| Mix | β-carotene (1.5% powder) | 712 | mg |
| | vitamin D3 powder (200000 IU/g) | 9 | mg |
| | vitamin E powder (20% powder) | 260 | mg |
| | vitamin K2 (0.2% powder) | 160 | mg |
| | thiamine hydrochloride | 14.88 | mg |
| | pyridoxine hydrochloride | 10.52 | mg |
| | riboflavin sodium phosphate | 10.32 | mg |
| | nicotinamide | 80 | mg |
| | calcium pantothenate | 55.2 | mg |
| | cyanocobalamin (0.1% powder) | 9.32 | mg |
| | folic acid | 3.2 | mg |
| | biotin yeast (50 mg/100 g) | 1068 | mg |

Water (1,420 g) was measured in a 5 L stainless bucket and heated to 70-80° C. Then, dextrin, granulated sugar, sodium ferrous citrate, sodium phosphate, potassium phosphate, potassium citrate and sodium gluconate were added to the water and dissolved by stirring in T. K. ROBOMICS (Tokushu Kika Kogyo Co., Ltd.) at 3,000 rpm. Edible fat and oil previously heated to 70-80° C. and an emulsifier were added thereto. Then, a source material for milk protein (Fonterra: casein content 66.8 wt %), monosodium glutamate monohydrate, mineral yeast Mix, and flavor were added in order and mixed to give a uniform solution/dispersion. Then, dissolved water-soluble food fiber, magnesium chloride, and calcium lactate were gradually added. Furthermore, vitamin Mix, sodium ascorbate and sodium erythorbate were added, dispersed, and dissolved. After adding water to a total weight of 4,000 g, the mixture was dissolved and dispersed until it reached a uniform state, and put in an aluminum pouch with a vent plug at 150 g per pouch. The pouch was subjected to a retort sterilization treatment by 2-step heating at 110° C. and 126° C.

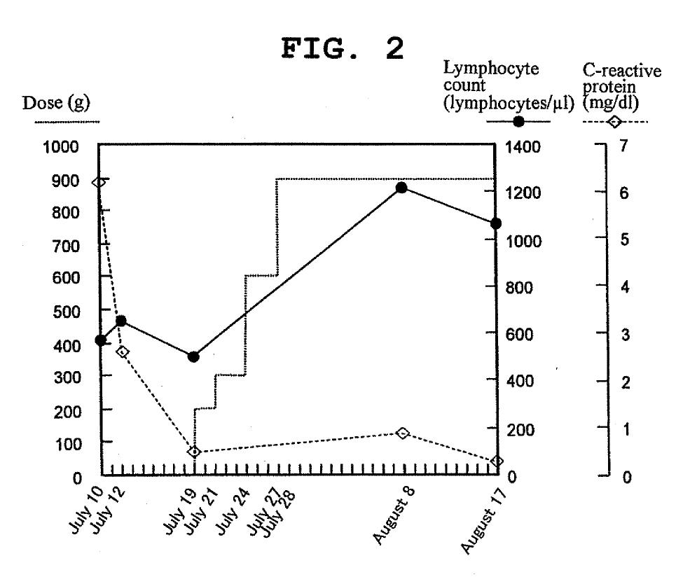

Since a hospitalized test subject (64 years old, male) developed aspiration pneumonia, the feeding method was changed from percutaneous endoscopic gastrostomy (PEG) without glutamic acid to intravenous hyperalimentation. However, the C-reactive protein value was high, and the test subject became repeatedly feverish. When the fever disappeared, the PEG agent with glutamic acid was administered in combination. The PEG agent with glutamic acid was intragastrically administered 3 times a day. The dose profile is shown in FIG. 2.

The profile of peripheral lymphocytes and the like after administration of the PEG agent with glutamic acid is as shown in FIG. 2. The peripheral lymphocyte count rose immediately after the administration and continued to remarkably increase during 3 weeks of continuous administration. At this time, the C-reactive protein value remained low and showed no shift.

### Example 3

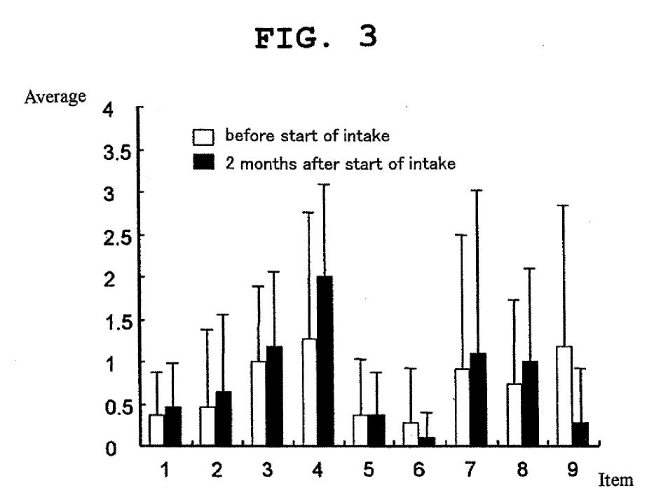

In the test of Example 1, the test subject was assessed by a nurse or a care assistant for the improvement of the general condition between immediately before the start of the administration of the rice gruel containing a salt of glutamic acid e and 2 months thereafter. The results are shown in Table 2. The test subjects took the Hasegawa Dementia Scale-Revised for the following 9 items immediately before the administration and 2 months thereafter. The results are shown in FIG. 3.

1. age?

2. what year, what month, what date, what day of the week?

3. where are you now?

4. memory of 3 words

5. continue to subtract 7 from 100

6. say numbers in reverse order

7. read out from memory of item 4

8. memory of 5 goods

9. say name of vegetables.

As is clear from Table 2, almost all test subjects showed a tendency toward improvement of mind and body conditions such as speech recovery, expression of emotion, and the like. In the Hasegawa Dementia Scale-Revised, the grade points increased particularly for the items relating to speech.

TABLE 2

| condition | | | | | | | | | | | | |
|---|---|---|---|---|---|---|---|---|---|---|---|---|
| observation | test subject No. | | | | | | | | | | | |
| items | 6598 | 7012 | 7020 | 6342 | 6990 | 5039 | 7082 | 7142 | 6957 | 5742 | 6362 | |
| Louder voice | ◯ | ◯ | | ◯ | | | ◯ | | | | | 4 |
| Clearer | ◯ | ◯ | | ◯ | | ◯ | ◯ | ◯ | ◯ | | | 7 |
| articulation | | | | | | | | | | | | |
| Use of voice | | ◯ | ◯ | ◯ | ◯ | | | | | | | 4 |
| Establishment of | ◯ | ◯ | | | | | | ◯ | ◯ | | | 4 |
| conversation | | | | | | | | | | | | |
| Recovery of memory | | ◯ | | ◯ | | | | | | | | 2 |
| Brighter facial | ◯ | ◯ | | ◯ | ◯ | ◯ | ◯ | | ◯ | ◯ | | 8 |
| expression | | | | | | | | | | | | |
| Outward facial | | ◯ | | ◯ | ◯ | | | ◯ | | ◯ | ◯ | 6 |
| expression | | | | | | | | | | | | |
| (delight, anger, | | | | | | | | | | | | |
| sorrow and | | | | | | | | | | | | |
| pleasure) | | | | | | | | | | | | |
| Recognition of | ◯ | ◯ | | | | | | | | | ◯ | 3 |
| meal | | | | | | | | | | | | |
| Eating by tightly | ◯ | ◯ | | | | | | | | | | 2 |
| holding a spoon | | | | | | | | | | | | |
| Eyes can be opened | ◯ | ◯ | ◯ | | ◯ | | | | | ◯ | ◯ | 6 |
| wide | | | | | | | | | | | | |
| 1 point for ◯, | 7 | 10 | 2 | 6 | 4 | 2 | 3 | 3 | 3 | 3 | 3 | |
| total points | | | | | | | | | | | | |
| (scale of one to | | | | | | | | | | | | |
| ten) | | | | | | | | | | | | |

Chronically compromised immunity in the elderly not only weakens the ability to defend against infectious diseases, but also causes general deterioration of the mind and body. The amount of lymphocytes in peripheral blood of the test subjects who took glutamic acid or a salt thereof increased as shown in Example 1, and immunity was enhanced. As a result, the condition of the mind and body can also be improved, namely, the overall quality of life can be improved.

### Production Example

To a powder (380 g) of monosodium glutamate monohydrate, which was passed through a 60 mesh sieve, are added lactose (883 g), which was passed through a 80 mesh sieve, and corn starch, which was passed through a 120 mesh sieve, and the mixture is thoroughly blended in a V-type mixer (powder A). Separately, to hydroxypropylcellulose (16 g), which was passed through a 60 mesh sieve, is added an appropriate amount of sterile purified water, and the mixture is dispersed at 90° C. and cooled to 20° C. (solution B). Solution B and powder A are kneaded in a biaxial kneading machine, and granulated in an extrusion-granulator (cylindrical shape, 0.5-1.0 mm). This is dried at 60° C. for 50 min in a through-flow dryer to give dry granules, which are sized in a tornado mill to give granules (1500 g).

1. A method of stimulating the immune system in a subject comprising administering an effective amount of glutamic acid or a salt thereof to said subject.

2. The method of claim 1, further comprising observing an increase in the lymphocytes present in the peripheral blood of said subject.

3. The method of claim 1, wherein the amount of glutamic acid or a salt thereof taken by said subject per day is 75-10,000 mg based on glutamic acid.

4. A method of increasing the lymphocytes in the peripheral blood of a subject, comprising administering an effective amount of glutamic acid or a salt thereof to said subject.

5. The method of claim 4, wherein the amount of glutamic acid or a salt thereof adminstered to said subject per day is 75-10,000 mg based on glutamic acid.

6. The method of claim 1, wherein the amount of glutamic acid or a salt thereof taken by said subject per day is 2,000-10,000 mg based on glutamic acid.

7. The method of claim 4, wherein the amount of glutamic acid or a salt thereof taken by said subject per day is 2,000-10,000 mg based on glutamic acid.

8. A composition comprising an immunostimulator and pharmaceutically acceptable diluents and/or excipients.

9. The composition of claim 8, which acts by increasing the lymphocytes in the peripheral blood when administered to a subject.

10. The composition of claim 8, wherein said immunostimulator comprises glutamic acid or a salt thereof.

11. The composition of claim 10, wherein said glutamic acid or salt thereof is present in an amount of about 75-10,000 mg.

12. The composition of claim 11, wherein said glutamic acid or salt thereof is present in an amount of about 2,000-10,000 mg.

13. A pharmaceutical composition comprising an immunostimulator and pharmaceutically acceptable excipients and/or diluents.

14. A food composition comprising an immunostimulator.

15. The food composition of claim 14, wherein the immunostimulator comprises glutamic acid or a salt thereof.

16. The food composition of claim 14, which is prepared and administered to a subject for health reasons.

17. A package comprising the food composition of claim 14 and written matter stating that the food composition is taken for the purpose of stimulating the immune system or increasing the lymphocytes in the peripheral blood, and which also explains the method of administration.
